(19) United States
(12) Patent Application Publication
(10) Pub. No.: US 2006/0183971 A1
(43) Pub. Date: Aug. 17, 2006

(54) Cellular telephone with remote video scope and motion detector

(76) Inventor: Yehuda Haviv, Ramat Gan (IL)

Correspondence Address: DR. MARK FRIEDMAN LTD.;C/o Bill Polkinghorn, 9003 Florin Way, Upper Marlboro, MD, 20772, US

(21) Appl. No.: 11/058,172

(22) Filed: Feb. 16, 2005

(51) Int. Cl. A61B 1/00; H04N 7/18

(52) U.S. Cl. 600/101; 600/109; 600/132; 348/74

(57) ABSTRACT

A cellular telephonic video scope and motion detector with the video scope and motion detector remotely and removably connected to the input/output port of the cellular telephone. A further embodiment allows control of the direction of the distal end of the video scope. A further embodiment allows attachment of the motion detector to the video scope. A further embodiment allows control of the video scope and motion detector via the cellular telephone keypad and cellular telephone transmission.

### FIELD AND BACKGROUND OF THE INVENTION

The present invention relates to the combination of a cellular telephone, a video scope, and a motion detector.

The use of endoscopic video cameras for various applications (predominantly medical uses) is well known in the art. The use of motion detectors for various applications (predominantly security related) is also well known in the art. The use of cellular telephony for various applications (predominantly communications) is also well known in the art. Video cameras and motion detectors have been used together in security-related applications. Cellular telephones with integrated video cameras have been used in the past. The use of a cellular telephone with a remote video scope and a motion detector as one device, however, has not yet been practiced.

The object of the present invention is to combine the video, illumination, mobility, and grasping and manipulative features of a video scope and the motion detecting features of a motion detector with the many features of a cellular telephone including a cellular telephone's input features (keypad), display features (screen), input/output features (I/O port), communications features, data storage features (memory), and data processing features (the cellular phone's central processing unit) in order to perform a variety of functions.

One limited embodiment of the present invention, using only a video camera on the distal end of a semi-rigid structural element, could be used to look into an enclosed space with an available or newly created lumen and display the contents of the enclosed space on the cellular telephone's screen. Another embodiment adding an illumination feature could be used in dark enclosed spaces. Another embodiment utilizing a motion detecting feature would allow the activation of the cellular telephone and the camera upon the recognition of motion in a monitored area and the transmission of the scene viewable by the stationary video camera. Still another higher level embodiment would allow movement of the video camera on the distal end of the flexible structural element by commands transmitted over the cellular telephone from a remote location.

All of these and other features of the present invention, some of which are discussed hereinbelow, have obvious uses in various applications in many fields including but not limited to the medical, mechanical monitoring and repair, and security fields. Therefore, there is a need for, and it would be advantageous to have, the present invention.

### SUMMARY OF THE INVENTION

According to a basic embodiment of the remote video input aspect of the present invention, there is provided a cellular telephone with a video display, a digital keypad, a speakerphone, and an input/output port and there is further provided a remote video scope apparatus. The cellular telephone is configured to accept video data input from the video scope apparatus and transmit control data back to the video scope apparatus through the cellular telephone input/output port. The cellular telephone is further configured to accept video apparatus control data from the cellular telephone keypad. The cellular telephone is further configured to accept video apparatus control data via cellular telephone transmission.

The video scope apparatus has a flexible though conformable and semi-rigid structural conduit member with a proximal end and a distal end. The proximal end of the video scope apparatus is fitted with a proximal video connector for the purpose of connecting the video scope apparatus to the input/output port of the cellular telephone. An image forming mechanism is attached at the video scope's distal end for the purpose of forming an image from light reflected or emitted from an object. The image forming mechanism is operatively connected to an image pickup mechanism for the purpose of converting the light formed into the image by the image forming mechanism into an electric signal. The image pickup mechanism is operatively connected to an electrical signal transmission mechanism for the purpose of transmitting the electric signal from the image pickup mechanism to the proximal video connector of the video scope apparatus. An electrical power transmission mechanism is connected in series from the proximal video connector of the video scope apparatus to the image pickup mechanism and image forming mechanism at the distal end for the purpose of providing electric power to the image pickup mechanism and image forming mechanism. The proximal video connector is conformed to plug into the cellular telephone input/output port for the purpose of transmitting electrical power from the cellular telephone through the electrical power transmission mechanism to the image pickup mechanism and image forming mechanism and for the purpose of transmitting the electric signal from the image pickup mechanism to the input/output port of the cellular telephone through the electrical signal transmission mechanism.

According to a further embodiment of the remote video data input aspect of the present invention, the proximal video connector is configured to allow the pass-through connection of additional devices to the same cellular telephone.

According to a further embodiment of the remote video data input aspect of the present invention, the proximal video connector is configured to allow connection to a variety of types of cellular telephone input/output ports.

According to a further embodiment of the remote video data input aspect of the present invention, there is provided an illumination ring around the outer circumference of the most distal aspect of the image forming mechanism operatively connected to the electrical power transmission mechanism for the purpose of illuminating the object being viewed by the image forming mechanism.

According to a further embodiment of the remote video data input aspect of the present invention, the structural conduit member is more flexible and less semi-rigid and there is provided a flexible tubular directional control sheath fitting over the exterior of the video scope apparatus for the purpose of controlling the direction of the distal end of the video scope apparatus. The length of the directional control sheath is approximately twenty centimeters shorter than the length of the entire video scope apparatus. The diameter of the distal and medial portions of the directional control sheath closely approximates the exterior diameter of the image forming mechanism and flares to a diameter of approximately ten centimeters at the proximal end of the directional control sheath. The directional control sheath terminates at its distal end with a distal directional control annulus that snugly fits over the exterior circumference of the image forming mechanism. The directional control sheath terminates at its proximal end with a proximal directional control annulus which loosely fits over the structural conduit member of the video scope apparatus approximately twenty centimeters distal from the proximal video connector. Four directional control attachment points are located on the proximal aspect of distal directional control annulus at ninety-degree intervals about the circumference of the distal directional control annulus. Four proximal directional control holes are located in the proximal directional control annulus at ninety-degree intervals aligned directly opposite the respective four directional control attachment points penetrating from the distal aspect of the proximal directional control annulus to the interior circumferential aspect of the proximal directional control annulus. Four tension control mechanisms are attached at the four directional control attachment points around the circumference of the proximal aspect of the distal directional control annulus at ninety-degree intervals, slidably run through the length of the directional control sheath and the four respective in the proximal directional control holes and terminate with four directional control finger rings.

According to a further embodiment of the remote video data input aspect of the present invention, the four directional control mechanisms terminate proximally to a manual joystick device attached to the proximal directional control annulus.

According to a further embodiment of the remote video data input aspect of the present invention, the directional control joystick device is configured with electrical directional controls.

According to a further embodiment of the remote video data input aspect of the present invention, the electrical directional controls are configured to respond to directional control commands transmitted through the keypad of the cellular telephone and the cellular telephone's input/output port.

According to a further embodiment of the remote video data input aspect of the present invention, the electrical directional controls are configured to respond to directional control commands transmitted from a remote telephone through the cellular telephone and the cellular telephone's input/output port.

According to a further embodiment of the remote video data input aspect of the present invention, there is provided a flexible multi-lumenal sheath removably and slidably fitting one of its lumena over the exterior of the video scope apparatus from the distal end of the proximal video connector to the most distal aspect of the video scope apparatus for the purpose of allowing the transmission of the video scope apparatus and instruments and objects through the remaining lumena to and from the respective proximal and distal ends of the lumena.

According to a basic embodiment of the motion detector aspect of the present invention, there is provided a cellular telephone with a video display, a digital keypad, a speakerphone, and an input/output port and there is further provided a remote motion detector apparatus. The cellular telephone is configured to accept motion detection data input from the motion detector apparatus and transmit control data back to the motion detector apparatus through the cellular telephone input/output port. The cellular telephone is further configured to accept motion detector control data from the cellular telephone keypad. The cellular telephone is further configured to accept motion detector control data via cellular telephone transmission. The motion detector apparatus has a flexible structural conduit member with a distal end and a proximal end. The proximal end of the motion detector apparatus is fitted with a proximal motion connector for the purpose of connecting the motion detector apparatus to the input/output port of the cellular telephone. A motion sensing mechanism is attached to the distal end of the motion detector's structural conduit for the purpose of sensing motion in the monitored area. The motion sensing mechanism is operatively connected to a motion pickup mechanism for the purpose of converting the signal from the motion sensing mechanism to electric signal. The motion pickup mechanism is operatively connected to an electrical signal transmission mechanism for the purpose of transmitting the electric signal from the motion pickup mechanism to the proximal motion connector. An electrical power transmission mechanism is connected in series from the proximal motion connector to the motion pickup mechanism and motion sensing mechanism for the purpose of transmitting electric power from the cellular telephone input/output port to the motion pickup mechanism and motion sensing mechanism.

According to a further embodiment of the motion detection aspect of the present invention, the proximal motion connector is configured to allow the pass-through connection of additional devices to the same cellular telephone.

According to a further embodiment of the motion detection aspect of the present invention, the proximal motion connector is configured to allow connection to a variety of types cellular telephone input/output ports.

According to a further embodiment of the motion detection aspect of the present invention, the distal end of the motion detector apparatus is adapted to attach astride the exterior of the image forming mechanism of the video scope apparatus.

According to a further embodiment of the motion detection aspect of the present invention, the motion detector apparatus is combined with the cellular telephone in one common case and the motion sensing mechanism and the motion pickup mechanism are directly attached to an electrical signal transmission mechanism and an electrical power transmission mechanism located in the cellular telephone.

The present invention discloses an innovative combination of a cellular telephone, a video scope, and a motion detector for a variety of functionality.

The use of the device is simple. The remote video apparatus is connected to the input/output port of the cellular telephone and the image sensed by the remote video apparatus is viewed on the display screen of the cellular telephone. This image may also be displayed on the display screen of other devices capable of receiving such images as digital transmission over cellular and physical transmission mechanisms. Using the pass-through connectors, the motion detector apparatus simultaneously may be attached to the same cellular telephone. The cellular telephone is configured to react to motion sensed by the motion detector apparatus by dialing a designated number, reporting the detection of motion, transmitting video data if it is available, and responding to instructions from the receiving unit. When motion is sensed by the motion detector apparatus, the cellular telephone dials the designated number, transmits the motion alarm and the available video data and corresponds with the receiver for further actions.

### BRIEF DESCRIPTION OF THE DRAWINGS

The invention is herein described, by way of example only, with reference to the accompanying drawings, wherein:

FIG. 1 illustrates the remote video aspect of the present invention;

FIG. 2 illustrates the proximal video connector configured for pass-through connection;

FIG. 3 illustrates the proximal video connector configured for an additional plurality of pass-through connectors;

FIG. 4 illustrates the illumination ring on distal end of the remote video apparatus;

FIG. 5 illustrates the flexible tubular directional control sheath;

FIG. 6 illustrates the termination of the four directional control tension control mechanisms to a manual directional control joystick;

FIG. 7 illustrates the direction control joystick device configured with electrical directional controls;

FIG. 8 illustrates electrical signal transmission mechanism is connected in series with the electrical directional controls;

FIG. 9 illustrates the device placed in the flexible quadralumenal sheath; and

FIG. 10 illustrates the motion detection aspect of the present invention;

### DESCRIPTION OF THE PREFERRED EMBODIMENTS

The present invention is of a cellular telephone adapted with a removable video scope and a removable motion detector, which can be used collectively for a variety of functionality. Specifically, the present invention can be used to monitor a location via a cellular telephone and to advise a user when motion is detected in the monitored location. The present invention can also be used to view otherwise inaccessible places using the cellular telephone display device, transmit the same image of the cellular telephone and control the positioning of the video scope using the cellular telephone keypad or cellular telephone transmission.

The principles and operation of a video scope and motion detector attached to a cellular telephone according to the present invention may be better understood with reference to the drawings and the accompanying description.

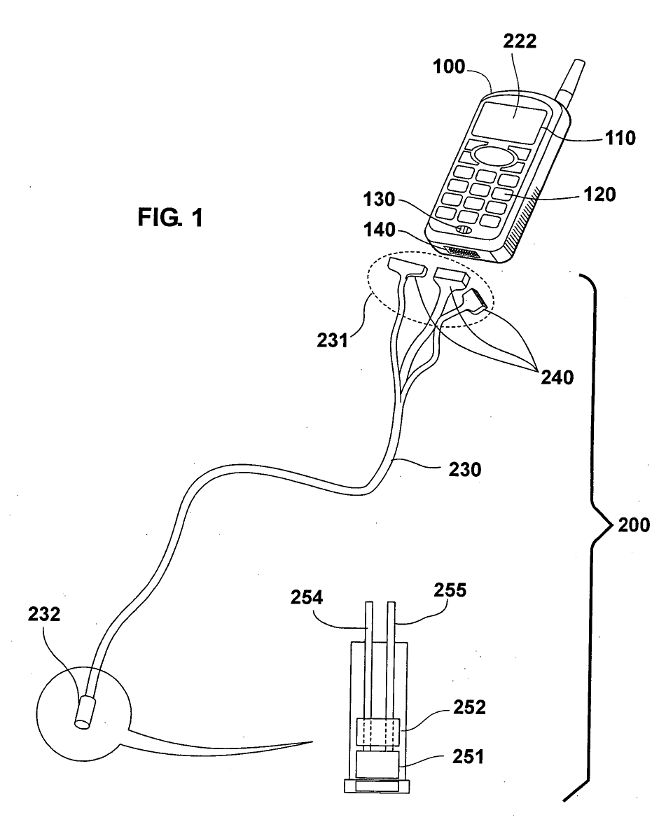

FIG. 1 illustrates a basic embodiment of one aspect of the present invention, wherein there is provided a cellular telephone 100 with a central processing unit 105 (not shown on drawing), a video display 110, a digital keypad 120, a speakerphone 130, and an input/output port 140 and there is further provided a remote video scope apparatus 200. The cellular telephone is configured to accept video data input 210 from the video scope apparatus 200 and transmit video scope apparatus control data 221 back to the video scope apparatus through the cellular telephone input/output port 140. The cellular telephone 100 is further configured to accept video scope apparatus keypad control data 222 from the cellular telephone keypad, convert such video scope apparatus keypad control data 222 to video scope apparatus control data 221 and transmit such video scope apparatus control data 221 to the video scope apparatus 200. The cellular telephone is further configured to accept remote video scope apparatus control data 223 via cellular telephone transmission, convert such remote video scope apparatus control data 223 to video scope apparatus control data 221 and transmit such video scope apparatus control data 221 to the video scope apparatus 200.

The video scope apparatus has a flexible though conformable and semi-rigid structural conduit member 230 with a proximal end 231 and a distal end 232. The proximal end of the video scope apparatus is fitted with a proximal video connector 240 for the purpose of connecting the video scope apparatus to the input/output port 140 of the cellular telephone. An image forming mechanism 251 is attached at the video scope's distal end 232 for the purpose of forming an image from light from an object. The image forming mechanism 251 is operatively connected to an image pickup mechanism 252 for the purpose of converting the light formed into the image by the image forming mechanism 251 into an electric signal 253. The image pickup mechanism is operatively connected to an electrical signal transmission mechanism 254 for the purpose of transmitting the electric signal from the image pickup mechanism 252 to the proximal video connector 240 of the video scope apparatus 200. An electrical power transmission mechanism 255 is connected in series from the proximal video connector 240 of the video scope apparatus 200 to the image pickup mechanism 252 and image forming mechanism 251 at the distal end 232 for the purpose of providing electric power 256 to the image pickup mechanism 252 and image forming mechanism 251. The proximal video connector 240 is conformed to plug into the cellular telephone input/output port 140 for the purpose of transmitting electrical power 256 from the cellular telephone 100 through the electrical power transmission mechanism 255 to the image pickup mechanism 252 and image forming mechanism 251, for the purpose of transmitting the electric signal 253 from the image pickup mechanism 252 to the input/output port 140 of the cellular telephone 100 through the electrical signal transmission mechanism 254 and for the purpose of transmitting video scope apparatus control data 221 to the image pickup mechanism 252 and the image forming mechanism 251.

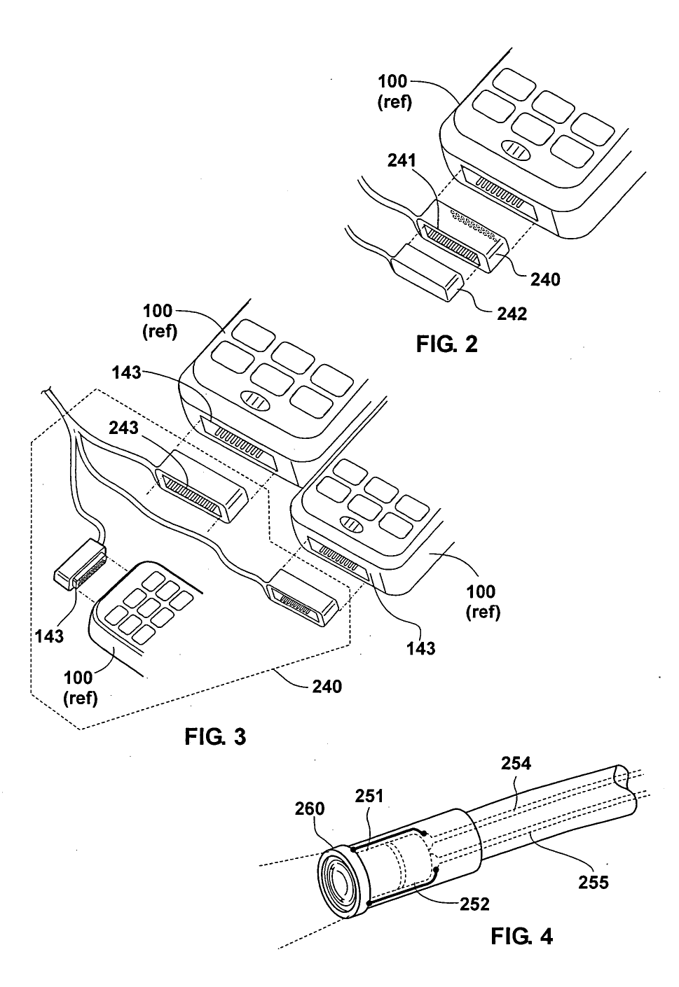

FIG. 2 illustrates a further embodiment of the present invention wherein the proximal video connector 240 is configured with a proximal video pass-through connector 241 to allow the pass-through connection of additional devices 242 to the same cellular telephone 100.

FIG. 3 illustrates a further embodiment of the present invention wherein the proximal video connector 240 is configured with an additional plurality of types of proximal video pass-through connectors 243 to allow connection to a plurality of types cellular telephone input/output ports 143.

FIG. 4 illustrates to a further embodiment of the present invention wherein there is provided an illumination ring 260 around the outer circumference of the most distal aspect of the image forming mechanism 251 operatively connected to the electrical power transmission mechanism 255 for the purpose of illuminating the object being viewed by the image forming mechanism 251.

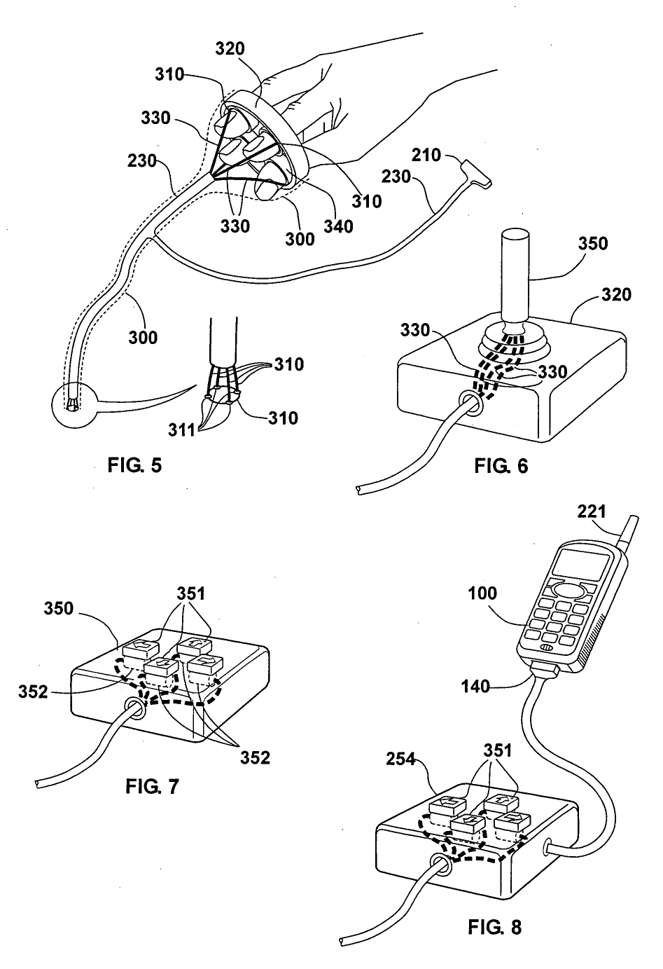

FIG. 5 illustrates a further embodiment of the present invention wherein the structural conduit member 230 is more flexible and less semi-rigid and there is provided a flexible tubular directional control sheath 300 fitting over the exterior of the video scope apparatus 200 for the purpose of controlling the direction of the distal end of the video scope apparatus. The length of the directional control sheath 300 is approximately twenty centimeters shorter than the length of the entire video scope apparatus 200. The diameter of the distal and medial portions of the directional control sheath closely approximates the exterior diameter of the image forming mechanism 251 and flares to a diameter of approximately ten centimeters at the proximal end of the directional control sheath 300. The directional control sheath terminates at its distal end with a distal directional control annulus 310 that snugly fits over the exterior circumference of the image forming mechanism 251 and terminates lo at the proximal end with a proximal directional control annulus 320 that loosely fits over the structural conduit member 230 of the video scope apparatus 200 approximately twenty centimeters distal from the proximal video connector 240. Four directional control attachment points 311 are located on the proximal aspect of distal directional control annulus 310 at ninety-degree intervals about the circumference of the distal directional control annulus. Four proximal directional control holes 321 in the proximal directional control annulus 320 are located at ninety-degree intervals aligned directly opposite the respective four directional control attachment points 311 penetrating from the distal aspect of the proximal directional control annulus 320 to the interior circumferential aspect of the proximal directional control annulus 320. Four directional control tension control mechanisms 330 are attached around the circumference of the proximal aspect of the distal directional control annulus 310 at ninety-degree intervals, slidably run through the length of the directional control sheath 300 and the four respective proximal directional control holes 321 in the proximal directional control annulus 320 and terminate with four directional control fingers rings 340.

FIG. 6 illustrates to a further embodiment of the present invention wherein the four directional control tension control mechanisms 330 terminate proximally to a manual directional control joystick device 350 attached to the proximal directional control annulus 320.

FIG. 7 illustrates a further embodiment of the present invention wherein, the directional control joystick device 350 is configured with electrical directional controls 351. The electrical directional controls 351 are operatively connected to directional control motors 352, which are in turn operatively connected to the four directional control tension control mechanisms 330, for the purpose of tightening and loosening the directional control tension control mechanisms 330 in response to pressure on the electrical directional controls 351 FIG. 8 illustrates a further embodiment of the present invention wherein the electric signal transmission mechanism 254 is additionally connected in series with the electrical directional controls 351, which are configured to respond to video scope apparatus control data 221 transmitted through the cellular telephone 100 and the cellular telephone's input/output port 140.

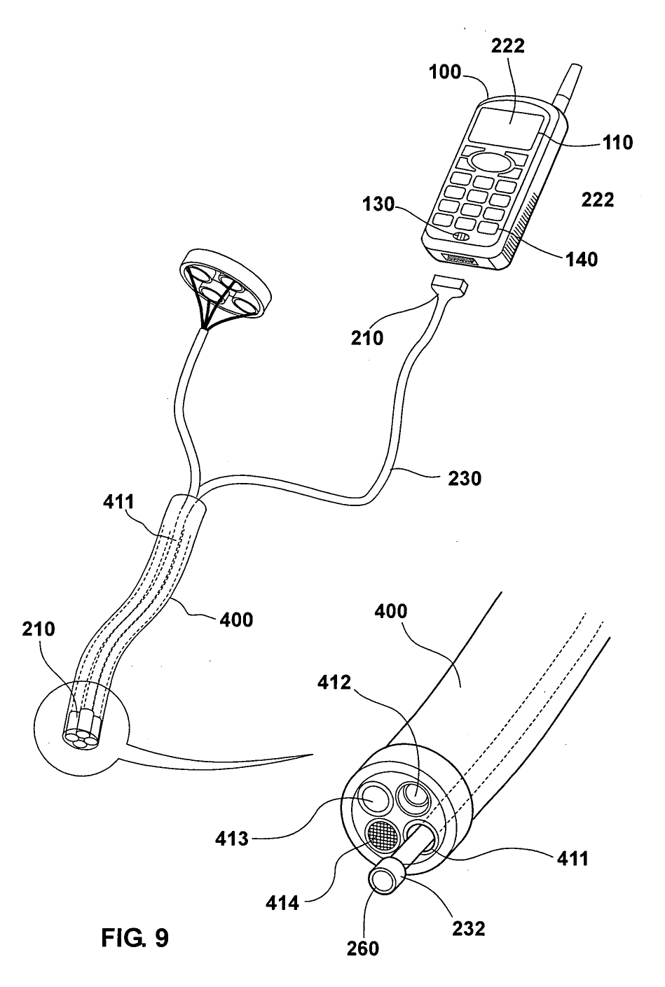

FIG. 9 illustrates a further embodiment of the present invention wherein there is provided a flexible quadralumenal sheath 400 removably and slidably fitting one of its four lumena 411 over the exterior of the video scope apparatus 200 from the distal end of the proximal video connector 240 to the most distal aspect of the video scope apparatus 200 for the purpose of allowing the transmission of the video scope apparatus and instruments and objects through the remaining three lumena 412, 413, 414, to and from the respective proximal and distal ends of the lumena.

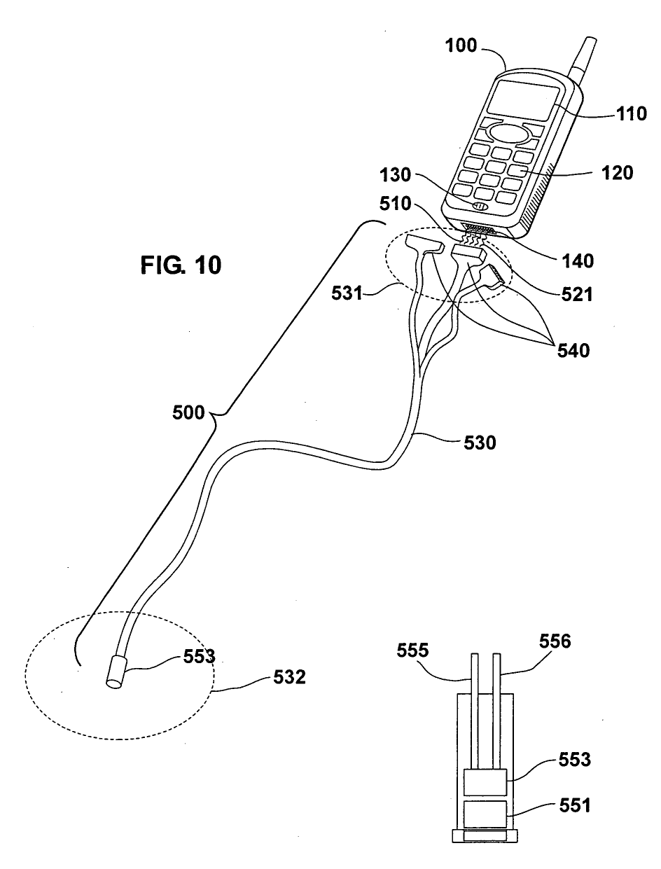

FIG. 10 illustrates a basic embodiment of another aspect of the present invention wherein there is provided a cellular telephone 100 with central processing unit 105, a video display 110, a digital keypad 120, a speakerphone 130, and an input/output port 140 and there is further provided a remote motion detector apparatus 500. The cellular telephone 100 is configured to accept motion detection data input 510 from the motion detector apparatus 500 and transmit motion detector control data 521 back to the motion detector apparatus through the cellular telephone input/output port 140. The cellular telephone 100 is further configured to accept keypad motion detector control data 522 from the cellular telephone keypad 120, convert such motion detector keypad control data 522 to motion detector control data 521 and transmit such motion detector control data 521 to the motion detector apparatus 500. The cellular telephone 100 is further configured to accept remote motion detector control data 523 via cellular telephone transmission, convert such remote motion detector control data 522 to motion detector control data 521 and transmit such motion detector control data 521 to the motion detector apparatus 500. The motion detector apparatus 500 has a flexible structural conduit member 530 with a proximal end 531 and a distal end 532. The proximal end 531 of the motion detector apparatus 500 is fitted with a proximal motion connector 540 for the purpose of connecting the motion detector apparatus 500 to the input/output port of the cellular telephone 140. A motion sensing mechanism 551 is attached to the distal end of the motion detector's structural conduit 530 for the purpose of sensing motion in the monitored area and reporting that motion as a motion sensing signal 552. The motion sensing mechanism 551 is operatively connected to a motion pickup mechanism 553 for the purpose of converting the motion sensing signal 552 from the motion sensing mechanism 551 to motion electric signal 554. The motion pickup mechanism 553 is operatively connected to a motion electrical signal transmission mechanism 555 for the purpose of transmitting the motion electric signal 554 from the motion pickup mechanism 553 to the proximal motion connector 540. An electrical power transmission mechanism 556 is connected in series from the proximal motion connector 540 to the motion pickup mechanism 553 and the motion sensing mechanism 551 for the purpose of transmitting electric power 557 from the cellular telephone input/output port 140 to the motion pickup mechanism 553 and the motion sensing mechanism 554.

While the invention has been described with respect to a limited number of embodiments, it will be appreciated that many variations, modifications and other applications of the invention may be made.

What is claimed is:

1. A cellular video scope device comprising: a cellular telephone connected to a remote video scope apparatus.

2. The device of claim 1 wherein the cellular telephone further comprises a central processing unit, a video display, a digital keypad, a speakerphone, and an input/output port.

3. The device of claim 2 wherein the cellular telephone is further configured to accept video data input from the video scope apparatus and transmit control data back to the video scope apparatus through the cellular telephone input/output port.

4. The device of claim 2 wherein the cellular telephone is further configured to accept video apparatus control data from the cellular telephone keypad.

5. The device of claim 2 wherein the cellular telephone is further configured to accept video apparatus control data via cellular telephone transmission.

6. The device of claim 2 wherein the video scope apparatus further comprises:
a. a structural conduit member with a proximal end and a distal end;
b. a proximal video connector attached to the proximal end of the video scope apparatus' structural conduit member for the purpose of connecting the video scope apparatus to the input/output port of the cellular telephone;
c. an image forming mechanism attached at the video scope's distal end for the purpose of forming an image from light from an object;
d. an image pickup mechanism operatively connected to the image forming mechanism for the purpose of converting the light formed into the image by the image forming mechanism into a video electric signal;
e. an electrical signal transmission mechanism operatively connected to the image pickup mechanism for the purpose of transmitting the video electric signal from the image pickup mechanism to the proximal video connector of the video scope apparatus; and
f. an electrical power transmission mechanism connected in series from the proximal video connector of the video scope apparatus to the image pickup mechanism and image forming mechanism for the purpose of providing electric power to the image pickup mechanism and image forming mechanism.

7. The device of claim 6 wherein the proximal video connector is further conformed to plug into the cellular telephone input/output port for the purpose of transmitting electrical power from the cellular telephone through the electrical power transmission mechanism to the image pickup mechanism and image forming mechanism and for the purpose of transmitting the electric signal from the image pickup mechanism to the input/output port of the cellular telephone through the electrical signal transmission mechanism.

8. The device of claim 6 wherein the proximal video connector is further configured with a duplicate of the telephone input/output port to which it is attached for the purpose of allowing pass-through connection of additional devices to the cellular telephone.

9. The device of claim 6 wherein the proximal video connector is further configured with a plurality of connectors for the purpose of allowing connection to a variety of types cellular telephone input/output ports.

10. The device of claim 6 further comprising an illumination ring around the outer circumference of the most distal aspect of the image forming mechanism operatively connected to the electrical power transmission mechanism and the electrical signal transmission mechanism for the purpose of illuminating the object being viewed by the image forming mechanism.

11. The device of claim 2 further comprising a directional control mechanism for the purpose of controlling the direction of the distal end of the video scope apparatus;

12. The device of claim 11, further comprising a directional control mechanism utilizing a manual joystick device.

13. The device of claim 11, further comprising a directional control joystick device configured with electrical directional controls and electrical directional control motors.

14. The device of claim 13 wherein the electrical directional controls are configured to respond to directional control commands transmitted through the cellular telephone.

15. The device of claim 1 further comprising a flexible multi-lumenal passage sheath removably and slidably fitting one of the passage sheath's lumena over the exterior of the video scope apparatus for the purpose of allowing the transmission of the video scope apparatus and instruments and objects through the remaining lumena to and from the respective proximal and distal ends of the lumena.

16. A cellular motion detector device comprising a cellular telephone connected to a remote motion detector apparatus.

17. The device of claim 16 wherein the cellular telephone further comprises a central processing unit, a video display, a digital keypad, a speakerphone, and an input/output port.

18. The device of claim 17 wherein the cellular telephone is further configured to accept motion detection data input from the motion detector apparatus and transmit motion detection control data back to the motion detector apparatus through the cellular telephone input/output port.

19. The device of claim 17 wherein the cellular telephone is further configured to accept motion detector control data from the cellular telephone keypad.

20. The device of claim 17 wherein the cellular telephone is further configured to accept motion detector control data via cellular telephone transmission.

21. The device of claim 17 wherein the remote motion detector apparatus further comprises
a. a flexible structural conduit member with a distal end and a proximal end;
b. a proximal motion connector connected to the proximal end of the motion detector apparatus for the purpose of connecting the motion detector apparatus to the input/output port of the cellular telephone;
c. a motion sensing mechanism attached to the distal end of the motion detector's structural conduit for the purpose of sensing motion in the monitored area and converting the motion into a motion signal;
d. a motion pickup mechanism operatively connected to the motion sensing mechanism for the purpose of converting the motion signal from the motion sensing mechanism to a motion electric signal;
e. an electrical signal transmission mechanism is operatively connected to the motion pickup mechanism for the purpose of transmitting the motion electric signal from the motion pickup mechanism to the proximal motion connector; and
f. an electrical power transmission mechanism connected in series from the proximal motion connector to the motion pickup mechanism and motion sensing mechanism for the purpose of transmitting electric power from the cellular telephone input/output port to the motion pickup mechanism and motion sensing mechanism.

22. The device of claim 21 wherein the proximal motion connector is further configured to allow the pass-through connection of additional devices to the same cellular telephone.

23. The device of claim 21 wherein the proximal motion connector is further configured to allow connection to a variety of types cellular telephone input/output ports.

24. The device of claim 16 wherein the distal end of the motion detector apparatus is further adapted to attach astride the exterior of an image forming mechanism of a video scope apparatus.

25. The device of claim 17 further comprising:
a. an internal motion detection electrical signal transmission connection point on the cellular telephone;
b. an internal motion detection electrical power transmission connection point on the cellular telephone;
c. a housing for the cellular telephone;
d. a motion sensing mechanism structurally attached to the housing of the cellular telephone and operatively attached to the exterior of the housing of the cellular telephone for the purpose of sensing motion in the monitored area and converting the motion into a motion signal;
e. a motion pickup mechanism structurally attached within the housing of the cellular telephone and operatively connected to the motion sensing mechanism for the purpose of converting the motion signal from the motion sensing mechanism to a motion electric signal;
f. an electrical signal transmission mechanism is operatively connected to the motion pickup mechanism and the internal motion detection electrical signal transmission connection point on the cellular telephone for the purpose of transmitting the motion electric signal from the motion pickup mechanism to the central processing unit of the cellular telephone; and
g. an electrical power transmission mechanism connected in series from the motion pickup mechanism and motion sensing mechanism and the internal motion detection electrical power transmission connection point on the cellular telephone for the purpose of transmitting electric power from the cellular telephone to the motion pickup mechanism and motion sensing mechanism.
